(19) United States
(12) Patent Application Publication
(10) Pub. No.: US 2026/0215724 A1
(43) Pub. Date: Jul. 30, 2026

(54) EARLY DETECTION OF COGNITIVE IMPAIRMENT

(71) Applicants: BOARD OF REGENTS, THE UNIVERSITY OF TEXAS SYSTEM, Austin, TX (US); THE BOARD OF SUPERVISORS OF LOUISIANA STATE UNIVERSITY AND AGRICULTURAL AND MECHANICAL COLLEGE, Baton Rouge, LA (US)

(72) Inventors: Robin C. HILSABECK, Austin, TX (US); Jeffrey KELLER, Baton Rouge, LA (US); Maya HENRY, Austin, TX (US); Paul TOPRAC, Austin, TX (US); Paul RATHOUZ, Austin, TX (US); Junyi LI, Austin, TX (US); Ray ALLEN, Baton Rouge, LA (US)

(73) Assignees: BOARD OF REGENTS, THE UNIVERSITY OF TEXAS SYSTEM, Austin, TX (US); THE BOARD OF SUPERVISORS OF LOUISIANA STATE UNIVERSITY AND AGRICULTURAL AND MECHANICAL COLLEGE, Baton Rouge, LA (US)

(21) Appl. No.: 19/148,555

(22) PCT Filed: Feb. 1, 2024
(86) PCT No.: PCT/US24/13983

§ 371 Date: Jul. 16, 2025

Related U.S. Application Data
(60) Provisional application No. 63/442,699, filed on Feb. 1, 2023.

(51) Int. Cl. A61B 5/00

(52) U.S. Cl. CPC A61B 5/4088; A61B 5/7275; A61B 5/7405; A61B 5/7475

(57) ABSTRACT

A computing system may include at least one processor and a memory. The computing system may be configured to: present a processing speed and working memory task to a user; receive first input from the user regarding the processing speed and working memory task; present a verbal task to the user; receive second input from the user regarding the verbal task; based on the first input and the second input, determine a cognitive performance score for the user; and provide a recommendation regarding care for the user based on the cognitive performance score.

### CROSS-REFERENCE TO RELATED APPLICATIONS

This application claims priority to U.S. Provisional Patent Application Ser. No. 63/442,699, filed Feb. 1, 2023, which is incorporated by reference herein in its entirety.

### STATEMENT REGARDING FEDERALLY SPONSORED RESEARCH OR DEVELOPMENT

This invention was made with government support under Grant No. R61 AG069780 awarded by the National Institutes of Health. The government has certain rights in the invention.

### TECHNICAL FIELD

The present disclosure relates in general to computing systems, and more particularly to the use of such systems in the early detection of cognitive impairment. Embodiments provide screening techniques that a primary care provider (PCP) may use to detect such impairment.

### BACKGROUND

Digital healthcare solutions that facilitate rapid identification and effective management of patients with Alzheimer disease and related disorders (ADRD) is an area of intense study. Because PCPs are the first line of medical care, they are often the first to hear concerns about cognitive decline. However, under-diagnosis of ADRD in the primary care settings is widely recognized, as are the many barriers to routine cognitive screening.

Early detection of cognitive decline is critical to the health and well-being of patients and their family members. In spite of the benefits of early detection, a significant percentage of individuals with cognitive impairment go undiagnosed. While primary care settings are an ideal place to screen for cognitive impairment in older adults, PCPs may lack confidence in their abilities to know who to screen, which brief assessment tool to use, and next steps that should be taken.

Embodiments of this disclosure thus provide a risk assessment and cognitive screening (RACS) application to facilitate identification of cognitive decline in primary care settings.

It should be noted that the discussion of a technique in the Background section of this disclosure does not constitute an admission of prior-art status. No such admissions are made herein, unless clearly and unambiguously identified as such.

### SUMMARY

In accordance with the teachings of the present disclosure, the disadvantages and problems associated with detection of cognitive impairment may be reduced or eliminated.

In accordance with embodiments of the present disclosure, a computing system may include at least one processor and a memory. The computing system may be configured to: present a processing speed and working memory task to a user; receive first input from the user regarding the processing speed and working memory task; present a verbal task to the user; receive second input from the user regarding the verbal task; based on the first input and the second input, determine a cognitive performance score for the user; and provide a recommendation regarding care for the user based on the cognitive performance score.

In accordance with these and other embodiments of the present disclosure, a method may include a computing system presenting a processing speed and working memory task to a user; the computing system receiving first input from the user regarding the processing speed and working memory task; the computing system presenting a verbal task to the user; the computing system receiving second input from the user regarding the verbal task; based on the first input and the second input, the computing system determining a cognitive performance score for the user; and the computing system providing a recommendation regarding care for the user based on the cognitive performance score.

In accordance with these and other embodiments of the present disclosure, an article of manufacture may include a non-transitory, computer-readable medium having computer-executable instructions thereon that are executable by a processor of a computing system for: presenting a processing speed and working memory task to a user; receiving first input from the user regarding the processing speed and working memory task; presenting a verbal task to the user; receiving second input from the user regarding the verbal task; based on the first input and the second input, determining a cognitive performance score for the user; and providing a recommendation regarding care for the user based on the cognitive performance score.

Technical advantages of the present disclosure may be readily apparent to one skilled in the art from the figures, description and claims included herein. The objects and advantages of the embodiments will be realized and achieved at least by the elements, features, and combinations particularly pointed out in the claims.

It is to be understood that both the foregoing general description and the following detailed description are examples and explanatory and are not restrictive of the claims set forth in this disclosure.

### BRIEF DESCRIPTION OF THE DRAWINGS

A more complete understanding of the present embodiments and advantages thereof may be acquired by referring to the following description taken in conjunction with the accompanying drawings, in which like reference numbers indicate like features, and wherein:

FIG. 1 illustrates a block diagram of an example computing system, in accordance with embodiments of the present disclosure; and

FIG. 2 illustrates an example method, in accordance with embodiments of the present disclosure;

FIG. 3 illustrates an example symbol matching task that may be used in a cognitive performance evaluation, in accordance with embodiments of the present disclosure; and

FIG. 4 illustrates an example speech language task that may be used in a cognitive performance evaluation, in accordance with embodiments of the present disclosure.

### DETAILED DESCRIPTION

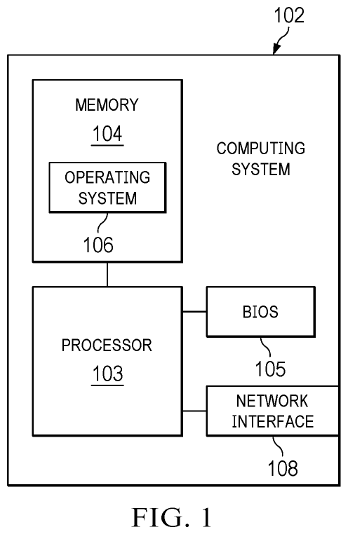

Preferred embodiments and their advantages are best understood by reference to FIGS. 1 through 4, wherein like numbers are used to indicate like and corresponding parts.

For the purposes of this disclosure, the term “computing system” may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, entertainment, or other purposes. For example, a computing system may be a personal computer, a personal digital assistant (PDA), a consumer electronic device such as a smartphone, a network storage device, or any other suitable device and may vary in size, shape, performance, functionality, and price. The computing system may include memory, one or more processing resources such as a central processing unit (“CPU”), and hardware or software control logic. Additional components of the computing system may include one or more storage devices, one or more communications ports for communicating with external devices as well as various input/output (“I/O”) devices, such as a keyboard, a mouse, and a video display. The computing system may also include one or more buses operable to transmit communication between the various hardware components.

For purposes of this disclosure, when two or more elements are referred to as “coupled” to one another, such term indicates that such two or more elements are in electronic communication or mechanical communication, as applicable, whether connected directly or indirectly, with or without intervening elements.

When two or more elements are referred to as “coupleable” to one another, such term indicates that they are capable of being coupled together.

For the purposes of this disclosure, the term “computer-readable medium” (e.g., transitory or non-transitory computer-readable medium) may include any instrumentality or aggregation of instrumentalities that may retain data and/or instructions for a period of time. Computer-readable media may include, without limitation, storage media such as a direct access storage device (e.g., a hard disk drive or floppy disk), a sequential access storage device (e.g., a tape disk drive), compact disk, CD-ROM, DVD, random access memory (RAM), read-only memory (ROM), electrically erasable programmable read-only memory (EEPROM), and/or flash memory; communications media such as wires, optical fibers, microwaves, radio waves, and other electromagnetic and/or optical carriers; and/or any combination of the foregoing.

FIG. 1 illustrates a block diagram of an example computing system 102, in accordance with embodiments of the present disclosure. In some embodiments, computing system 102 may comprise a server chassis configured to house a plurality of servers or “blades.” In other embodiments, computing system 102 may comprise a personal computer (e.g., a desktop computer, laptop computer, mobile computer, and/or notebook computer). In yet other embodiments, computing system 102 may comprise a storage enclosure configured to house a plurality of physical disk drives and/or other computer-readable media for storing data (which may generally be referred to as “physical storage resources”). As shown in FIG. 1, computing system 102 may comprise a processor 103, a memory 104 communicatively coupled to processor 103, a BIOS 105 (e.g., a UEFI BIOS) communicatively coupled to processor 103, a network interface 108 communicatively coupled to processor 103. In addition to the elements explicitly shown and described, computing system 102 may include one or more other computing resources.

Processor 103 may include any system, device, or apparatus configured to interpret and/or execute program instructions and/or process data, and may include, without limitation, a microprocessor, microcontroller, digital signal processor (DSP), application specific integrated circuit (ASIC), or any other digital or analog circuitry configured to interpret and/or execute program instructions and/or process data. In some embodiments, processor 103 may interpret and/or execute program instructions and/or process data stored in memory 104 and/or another component of computing system 102.

Memory 104 may be communicatively coupled to processor 103 and may include any system, device, or apparatus configured to retain program instructions and/or data for a period of time (e.g., computer-readable media). Memory 104 may include RAM, EEPROM, a PCMCIA card, flash memory, magnetic storage, opto-magnetic storage, or any suitable selection and/or array of volatile or non-volatile memory that retains data after power to computing system 102 is turned off.

As shown in FIG. 1, memory 104 may have stored thereon an operating system 106. Operating system 106 may comprise any program of executable instructions (or aggregation of programs of executable instructions) configured to manage and/or control the allocation and usage of hardware resources such as memory, processor time, disk space, and input and output devices, and provide an interface between such hardware resources and application programs hosted by operating system 106. In addition, operating system 106 may include all or a portion of a network stack for network communication via a network interface (e.g., network interface 108 for communication over a data network). Although operating system 106 is shown in FIG. 1 as stored in memory 104, in some embodiments operating system 106 may be stored in storage media accessible to processor 103, and active portions of operating system 106 may be transferred from such storage media to memory 104 for execution by processor 103.

Network interface 108 may comprise one or more suitable systems, apparatuses, or devices operable to serve as an interface between computing system 102 and one or more other computing systems via an in-band network. Network interface 108 may enable computing system 102 to communicate using any suitable transmission protocol and/or standard. In these and other embodiments, network interface 108 may comprise a network interface card, or “NIC.” In these and other embodiments, network interface 108 may be enabled as a local area network (LAN)-on-motherboard (LOM) card.

As discussed above, embodiments of this disclosure may provide an automated risk assessment and cognitive screening (RACS) application (or app) that is easy to use for both PCPs and patients who may have cognitive impairment. Various portions of the RACS application may be implemented via one or more computing systems 102. An assessment via the RACS app may determine a likelihood that a patient has mild cognitive impairment (MCI), dementia, Alzheimer's, etc.

Embodiments of this disclosure may be implemented as an application that may be executed on a smart phone or other computing system. Other embodiments may be implemented as a web page, a progressive web app (PWA), etc. The computing system used for implementing embodiments may include one or more interfaces for receiving the user's responses to the tasks (e.g., a touch interface and/or mouse and/or keyboard for working memory tasks, a microphone for speech-language tasks, a camera for certain tasks, etc.).

In some embodiments, the application may be self-contained. In other embodiments, the application may communicate over a network (e.g., the internet) to one or more remote computing systems. For example, the local system may provide an interface for the user, while the remote system may provide the tasks to be completed by the patient and/or receive information regarding the results of such tasks and/or process the results of such tasks to provide recommendations for the patient.

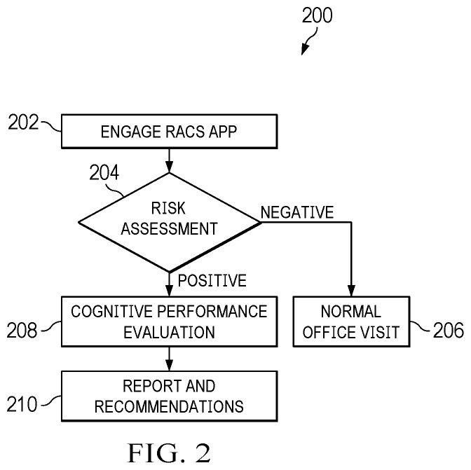

Turning now to FIG. 2, a flow chart of an example method 200 is shown for patient screening with a RACS app, according to some embodiments.

At step 202, a patient may begin using the RACS app. Patients in general may include healthy patients, patients with MCI, patients with dementia, etc., and the RACS app may be operable to categorize patients according to their level of cognitive impairment. The RACS app may first ask for various information to enroll the patient (e.g., name, date of birth, etc.).

At step 204, the RACS app may carry out a risk assessment. If the risk assessment is negative, the patient is determined to have a likelihood of healthy cognitive function, and may proceed for a normal office visit at step 206.

If the risk assessment is positive, the patient is determined to be part of a population that has high-risk for cognitive impairment. At step 208, the RACS app may carry out a cognitive performance evaluation, including a personal memory test, a symbol matching test, and/or any other suitable test. The results of the cognitive performance evaluation may be compared statistically with historical results to determine whether or not the patient has a likelihood (e.g., a 50% likelihood or any other suitable threshold) of MCI or dementia.

At step 210, the RACS app may provide a report with recommendations (e.g., details about recommended further treatment, etc.) to the patient, to the patient's PCP, or to any other suitable party.

One of ordinary skill in the art with the benefit of this disclosure will understand that the preferred initialization point for the method depicted in FIG. 2 and the order of the steps comprising the method may depend on the implementation chosen. In these and other embodiments, the method may be implemented as hardware, firmware, software, applications, functions, libraries, or other instructions. Further, although FIG. 2 discloses a particular number of steps to be taken with respect to the disclosed method, the method may be executed with greater or fewer steps than depicted. The method may be implemented using any of the various components disclosed herein (such as the components of FIG. 1), and/or any other system operable to implement the method.

Thus in one embodiment, the RACS application may provide three components: (1) a risk assessment based on questions about health history; (2) a cognitive performance evaluation, which may be measured by processing speed/working memory and speech-language tasks; and (3) individualized follow-up recommendations.

In one embodiment, the risk assessment may include various questions regarding the patient's health history having answers that correlate with a risk of cognitive impairment, such as:
Have you ever had a stroke?
Do you have any concerns about your memory?
Does someone you know have concerns about your memory?
Did you finish high school or obtain a GED?
What is your height?
What is your weight?
Do you have type 2 diabetes?
Do you need help from others to manage money or medications?
Do you take antidepressant medication?
In the past week, how many days did you feel that everything was an effort?

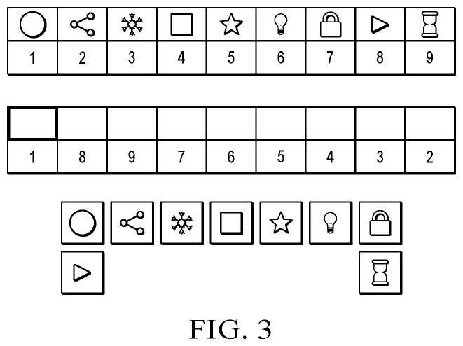

The cognitive performance evaluation may include various tasks, such as symbol matching tasks to determine processing speed and/or working memory retention. FIG. 3 shows an example symbol matching task in which the patient needs to match specific symbols with specific numbers. For example, to complete the task shown in FIG. 3, the patient would tap the circle button for number 1, the arrow button for number 8, and so on. The user may have a defined time limit (e.g., 90 seconds) to complete the symbol matching task, and the RACS app may present a series of such tasks up to the time limit to judge how quickly the patient can complete them.

The cognitive performance evaluation may also include speech language tasks to be performed orally, such as counting forward or backward between designated numbers, recounting a story about an important event, explaining how to complete a task such as making a peanut butter and jelly sandwich, describing a picture, etc. The tasks that are performed orally may be recorded via a microphone and evaluated either immediately by the RACS app (e.g., via the use of text-to-speech and/or artificial intelligence techniques) or later by a human evaluator.

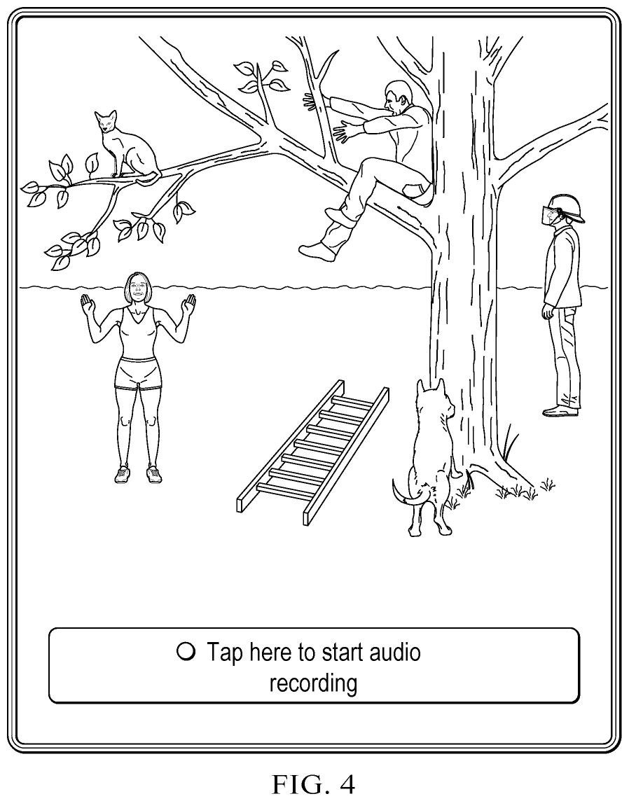

FIG. 4 shows one example of such a task for describing a picture. The patient is shown a picture that may illustrate a story. The patient is instructed to verbally tell a story having a beginning, a middle, and an end corresponding to the picture.

The oral tasks may then be analyzed with a custom, HIPAA-compliant speech-language analysis pipeline to extract acoustic features (all samples) and linguistic features (those connected speech tasks only) for each audio sample. In one embodiment, a specific set of acoustic and linguistic features may be used as predictors of cognitive impairment, in addition to symbol matching performance (SM) and differences in performance between subsequent runs of symbol matching tests (SM2-SM difference).

The acoustic variable set comprises voice parameters (e.g., mean and variability of fundamental frequency, mean and variability of formant frequencies, and jitter) derived using a tool such as openSMILE (open-source Speech and Music Interpretation by Large-space Extraction), and speech timing measures (e.g., speech rate and articulation rate) derived using a tool such as Praat software. After transcription of the audio file using an automatic speech recognition tool, the linguistic features may be derived using tools such as SpaCy and SPLAT.

Linguistic features generally comprise lexical-semantic variables (e.g., word frequency, prevalence, semantic diversity, concreteness, and age of acquisition) as well as morpho-syntactic variables (e.g., noun-verb ratio, normalized part of speech counts, and grammatical complexity indices).

In particular, the following list of acoustic features were selected:
F0semitoneFrom27.5 Hz_sma3nz_amean
F0semitoneFrom27.5 Hz_sma3nz_stddevNorm
F0semitoneFrom27.5 Hz_sma3nz_percentile20.0
F0semitoneFrom27.5 Hz_sma3nz_percentile50.0
F0semitoneFrom27.5 Hz_sma3nz_percentile80.0
F0semitoneFrom27.5 Hz_sma3nz_pctlrange0.2
F0semitoneFrom27.5 Hz_sma3nz_meanRisingSlope
F0semitoneFrom27.5 Hz_sma3nz_stddevRisingSlope
F0semitoneFrom27.5 Hz_sma3nz_meanFallingSlope
F0semitoneFrom27.5 Hz_sma3nz_stddevFallingSlope
jitterLocal_sma3nz_amean
jitterLocal_sma3nz_stddevNorm
F1frequency_sma3nz_amean
F1frequency_sma3nz_stddevNorm
F2frequency_sma3nz_amean
F2frequency_sma3nz_stddevNorm
F3frequency_sma3nz_amean
F3frequency_sma3nz_stddevNorm
VoicedSegmentsPerSec
Mean VoicedSegmentLengthSec
StddevVoicedSegmentLengthSec
MeanUnvoicedSegmentLength
StddevUnvoicedSegmentLength
speechrate.nsyll . . . dur.
articulation.rate.nsyll . . . phonationtime.
Speech.to.pause.ratio

Further, the following list of linguistic features were selected:
Grammar.Complexity. Index (the number of complex grammar relations as a fraction of the number of grammar relations, as determined by SpaCy's dependency parser)
Noun.to. Verb.ratio
# . . . of.unique.words
Type.Token.Ratio (number of unique words as a fraction of the number of words)
Moving.Average.Type.Token.Ratio . . . n.5 (Type token ratio applied with a moving window of 5 words)
Moving.Average.Type.Token.Ratio . . . n.15 (Type token ratio applied with a moving window of 15 words)
Propositional.Density (percentage of words labeled with tags such as verb, adjective, adverb, preposition, and conjunction)
Part-of-speech tag counts (normalized by the number of tags in the sample) for each part of speech:
   POS_TAG.ADJ
   POS_TAG.ADP
   POS_TAG.ADV
   POS_TAG.AUX
   POS_TAG.DET
   POS_TAG.INTJ
   POS_TAG.NOUN
   POS_TAG.NUM
   POS_TAG.PART
   POS_TAG.PRON
   POS_TAG.PROPN
   POS_TAG.VERB
   POS_TAG.CONJ
Fillers.per.word
Repetitions.per.word
content.function.ratio
Mean.Word.Frequency
Mean.Word.Length.By.Phonemes
Mean.Word.Semantic.Diversity (words that appear in a wide range of contexts on diverse topics are more variable in meaning than those that appear in a restricted set of similar contexts)
Mean.Word.Prevalence (number of people who know a given word)
Mean.Word.Concreteness (degree to which the concept denoted by a word refers to a perceptible entity)
Mean.Word.Age.of.Acquisition
Mean.Word.Length.by.Morphemes

Bayesian adaptive regression trees (BART) is a tree-based ensemble learning approach for prediction and classification. Rather than learning (fitting) a single classification or regression tree, BART fits many such trees and averages the results to get a more accurate classifier. In addition, BART operates in a Bayesian framework, facilitating posterior inferences about specific model features. For each model, leave-two-out cross-validation (CV) may be used to select two BART tuning parameters: the number of trees, and a parameter controlling the smoothness of each tree. In the leave-two-out CV, one each of a cognitively normal (CN) and a cognitively impaired (CI) participant were excluded from the data, the model was fitted, and external predicted values were computed for the left-out pair. The resulting c-statistic (or, equivalently, the area under the ROC curve) is the average number of times the fitted pair of predictions was concordant with the actual cognitive status of the held-out (external) pair.

The estimated external c-statistic may be used to choose among candidate models. Within each model, the importance of any given variable may be assessed as the proportion of all branch splits in that model accounted for by that variable. In addition, for selected variables, a Bayesian “p-value” may be computed as the posterior probability of a monotonic (non-zero) trend in the partial dependence plot (PDP) of the classifier on that variable, where the PDP is a non-linear analogue of an adjusted effect. Because these will be one-sided probabilities, a posterior probability of <0.025 may be used as a threshold for nominal significance.

According to one embodiment, 11 models in total were considered. All models contained sex, age, education, and marital status as demographic predictors. One model contained just SM and the SM2-SM difference. Four models contained the acoustic and linguistic variables discussed above from each of the four tasks (although counting backwards did not have linguistic features), and four more models were identical, except with the two SM variables added in.

Finally, for comparison, two models with quick MCI (QMCI) were fitted: one with and one without the two SM variables. The top four models all included the two SM variables: the one with SM by itself (estimated c=0.91), one with SM and features from the personal memory task (c=0.94), and one with SM and the counting backwards task (c=0.90). Models with the picture description and procedural discourse tasks performed the worst, and these tasks were not considered further. In the model with just the two SM tasks, the SM and SM2-SM difference scores were the two most important variables, and the Bayesian PDP p-values in this model were ~0.001 and 0.055.

In the model with the personal memory task, p-values were 0.21 and 0.53, but it is possible that there is overlap in signal between the SM variables and the personal memory variables. Finally, as a comparison, the QMCI-only model yielded c=0.91.

In general, embodiments may use a combination of working memory/processing speed, acoustic and linguistic variables involved in recounting personal memories, and the ability to benefit from prior exposure to a task to discriminate cognitively normal from cognitively impaired groups with a high degree of accuracy. This unique combination assesses key cognitive abilities known to be affected early in a variety of neurodegenerative diseases, i.e., executive functioning and memory, and includes both verbal and nonverbal tasks, which may be an important feature for individuals who are not able to do one type of task or the other, such as when patients have speech production or visual deficits. Importantly, individuals with varying levels of cognitive functioning, including mild dementia, are able to use the RACS app successfully, and the automated nature of the tool limits the amount of staff time and expertise needed.

In one embodiment, the RACS app may include only the SM and personal memory tasks and may generate a cognitive performance (CP) score derived from the relevant features of these tasks. Follow-up instructions based on the CP score may be provided to the primary care provider, including specific verbiage for communicating results of the screening test to the patient, recommendations for next steps if further evaluation is indicated, and handouts to provide patients explaining benefits of early diagnosis, what to expect as part of a more comprehensive work-up, and available community resources.

Although various possible advantages with respect to embodiments of this disclosure have been described, one of ordinary skill in the art with the benefit of this disclosure will understand that in any particular embodiment, not all of such advantages may be applicable. In any particular embodiment, some, all, or even none of the listed advantages may apply.

This disclosure encompasses all changes, substitutions, variations, alterations, and modifications to the exemplary embodiments herein that a person having ordinary skill in the art would comprehend. Similarly, where appropriate, the appended claims encompass all changes, substitutions, variations, alterations, and modifications to the exemplary embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative.

Unless otherwise specifically noted, articles depicted in the drawings are not necessarily drawn to scale. However, in some embodiments, articles depicted in the drawings may be to scale.

Further, reciting in the appended claims that a structure is “configured to” or “operable to” perform one or more tasks is expressly intended not to invoke 35 U.S.C. § 112 (f) for that claim element. Accordingly, none of the claims in this application as filed are intended to be interpreted as having means-plus-function elements. Should Applicant wish to invoke § 112 (f) during prosecution, Applicant will recite claim elements using the “means for [performing a function]” construct.

All examples and conditional language recited herein are intended for pedagogical objects to aid the reader in understanding the invention and the concepts contributed by the inventor to furthering the art, and are construed as being without limitation to such specifically recited examples and conditions. Although embodiments of the present inventions have been described in detail, it should be understood that various changes, substitutions, and alterations could be made hereto without departing from the spirit and scope of the disclosure.

What is claimed is:

1. A computing system comprising:
at least one processor; and
a memory;
wherein the computing system is configured to:
present a processing speed and working memory task to a user;
receive first input from the user regarding the processing speed and working memory task;
present a verbal task to the user;
receive second input from the user regarding the verbal task;
based on the first input and the second input, determine a cognitive performance score for the user; and
provide a recommendation regarding care for the user based on the cognitive performance score.

2. The computing system of claim 1, further configured to present medical screening questions to the user, wherein the recommendation is further based on answers to the questions.

3. The computing system of claim 1, wherein the processing speed and working memory task comprises a speed-based symbol matching task, and wherein the computing system is further configured to receive the first input via a touch interface.

4. The computing system of claim 1, wherein the verbal task comprises recalling a personal event.

5. The computing system of claim 1, wherein the cognitive performance score is determined based on a Bayesian adaptive regression trees (BART) analysis.

6. The computing system of claim 5, further configured to derive a plurality of linguistic features from the second input, wherein the BART analysis is based on the derived plurality of linguistic features.

7. The computing system of claim 5, further configured to derive a plurality of acoustic features from the second input, wherein the BART analysis is based on the derived plurality of acoustic features.

8. A method comprising:
a computing system presenting a processing speed and working memory task to a user;
the computing system receiving first input from the user regarding the processing speed and working memory task;
the computing system presenting a verbal task to the user;
the computing system receiving second input from the user regarding the verbal task;
based on the first input and the second input, the computing system determining a cognitive performance score for the user; and
the computing system providing a recommendation regarding care for the user based on the cognitive performance score.

9. The method of claim 8, further comprising presenting medical screening questions to the user, wherein the recommendation is further based on answers to the questions.

10. The method of claim 8, wherein the processing speed and working memory task comprises a speed-based symbol matching task, and wherein the method further comprises receiving the first input via a touch interface.

11. The method of claim 8, wherein the verbal task comprises recalling a personal event.

12. The method of claim 8, wherein the cognitive performance score is determined based on a Bayesian adaptive regression trees (BART) analysis.

13. The method of claim 12, further comprising deriving a plurality of linguistic features from the second input, wherein the BART analysis is based on the derived plurality of linguistic features.

14. The computing system of claim 12, further comprising deriving a plurality of acoustic features from the second input, wherein the BART analysis is based on the derived plurality of acoustic features.

15. An article of manufacture comprising a non-transitory, computer-readable medium having computer-executable instructions thereon that are executable by a processor of a computing system for:
presenting a processing speed and working memory task to a user;
receiving first input from the user regarding the processing speed and working memory task;
presenting a verbal task to the user;
receiving second input from the user regarding the verbal task;
based on the first input and the second input, determining a cognitive performance score for the user; and
providing a recommendation regarding care for the user based on the cognitive performance score.

16. The article of claim 15, wherein the instructions are further executable for presenting medical screening questions to the user, wherein the recommendation is further based on answers to the questions.

17. The article of claim 15, wherein the processing speed and working memory task comprises a speed-based symbol matching task, and wherein the computing system is further configured to receive the first input via a touch interface.

18. The article of claim 15, wherein the verbal task comprises recalling a personal event.

19. The article of claim 15, wherein the cognitive performance score is determined based on a Bayesian adaptive regression trees (BART) analysis.

20. The article of claim 19, wherein the instructions are further executable for deriving a plurality of linguistic features from the second input, wherein the BART analysis is based on the derived plurality of linguistic features.

21. The article of claim 19, wherein the instructions are further executable for deriving a plurality of acoustic features from the second input, wherein the BART analysis is based on the derived plurality of acoustic features.
